(19) United States
(12) Patent Application Publication
(10) Pub. No.: US 2010/0317916 A1
(43) Pub. Date: Dec. 16, 2010

(54) Method for relative quantitation of chromosomal DNA copy number in single or few cells

(76) Inventors: Richard T. Scott, JR., Far Hills, NJ (US); Nathan R. Treff, Chester, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/802,738

(22) Filed: Jun. 11, 2010

Related U.S. Application Data
(60) Provisional application No. 61/268,483, filed on Jun. 12, 2009.

(51) Int. Cl. A61B 17/43; C12Q 1/68; C40B 40/06; C40B 50/14

(52) U.S. Cl. 600/35; 435/6; 506/16; 506/30

(57) ABSTRACT

The present invention is directed to methods for determining the presence or absence of a genetic defect in an IVF embryo prior to transfer comprising performing real-time PCR and 2^{-ΔΔC}_{T} analyses to determine normalized copy number of at least one invariant locus on at least one chromosome collected from at least one cell of the embryo and selecting a candidate IVF embryo determined to be without genetic defect for transfer.

This application claims the benefit of the filing date of U.S. Provisional Patent Application No. 61/268,483 filed Jun. 12, 2009, the disclosure of which is hereby incorporated herein by reference.

### BACKGROUND OF THE INVENTION

For several decades many couples have been treated for infertility using the technique of in vitro fertilization (IVF). This procedure involves the in vitro incubation of sperm and an egg in culture media in which fertilization takes place. The fertilized egg is then cultured in special media for several days before the embryo is transferred into the female patient.

Typically, embryos are cultured for 3 days prior to transfer. It is also clinically possible to culture IVF embryos for several more days during which time the embryo develops into a blastocyst. Delaying embryo transfer until day 5 is thought to result in a greater chance of implantation, thus clinicians need not transfer as many embryos as might be typically transferred on day 3, thus reducing the possibility of a high risk multiple pregnancy. In some cases, embryos may be transferred on day 6, as some blastocysts may develop more slowly than others, but are still reproductively competent.

Preimplantation genetic diagnosis (PGD) may be used to screen IVF embryos for genetic defects, or otherwise grade the embryo's viability, prior to embryo transfer. Of the possible genetic defects, aneuploidy is the most prevalent genetic abnormality in human embryos derived through in vitro fertilization. By identifying embryos with chromosomal abnormalities such as aneuploidy, PGD can be used to avoid transferring embryos which may fail to implant or which may eventually end in a miscarried pregnancy. Using PGD to determine the presence of chromosomal abnormalities in an IVF embryo prior to transfer can also ease the minds of individuals with a family history of genetic disease and who fear passing on a genetic abnormality to their child.

PGD involves the analysis of nucleic acid derived from cells removed from an IVF embryo during the preimplantation stage of development. While biopsy of first polar bodies prior to fertilization or second polar bodies after fertilization on day 1 is possible, typically, PGD is performed using nucleic acid isolated from a single cell from a day 3 embryo. At least one healthy embryo identified by genetic analysis can then be transferred. If the embryo is to be transferred before day 5 (or day 6, in some cases), the embryos need not be frozen.

US 2008/0243398 and related application, 2007/0184467 (Rabinowitz et al.) describe a mathematical protocol for cleansing noisy genetic data and determining chromosome copy number. The techniques disclosed in these references involve assay of the genotype of one or more fertilized embryos as well as of the parents or other related individuals. Through sophisticated mathematical filtering, the genomes are compared in order to reconstruct the incomplete genetic data obtained from the embryo with the data obtained from the parents or related individuals to permit analysis of chromosome copy number in the embryo or to make phenotypic predictions. However, this technique involves whole genome analysis of the embryo, parents and/or other related individuals, the creation of data which may contain significant amplification errors, as well as the mathematical manipulation of a considerable volume of data. (See also, Johnson, D. S. et al., Fertility and Sterility, Vol. 90, Suppl 1, September 2008, pp. S309-S310; Rabinowitz, M. et al., Fertility and Sterility, Vol. 90, Suppl 1, September 2008, p. S23; and Johnson, D. M. et al., Fertility and Sterility, Vol. 89, Issue 4, p. S5).

U.S. Pat. No. 7,442,506 and U.S. Pat. No. 7,332,277 disclose methods for screening a fetus at multiple loci of interest associated with a trait or disease state to detect genetic disorders in a fetus.

As understood by one of skill in the art, the real time polymerase chain reaction (RT-PCR) is a conventional tool of molecular biology which is used to amplify and quantify a target DNA molecule in a sample. The amount of DNA may be determined as an absolute copy number or as a relative amount. Specifically, the use of RT-PCR to quantify gene expression using the comparative C_{T} method is familiar to one of skill in the art. (See, e.g., Schmittgen, T. and Livak, K. Nature Protocols, Vol. 3, No. 6, pp 1101-1108, (2008)).

In general, the threshold cycle (C_{T}) for a given genetic locus may be determined by arbitrarily setting a signal intensity threshold that falls within the linear range of amplification of real time PCR data. Previous application of this calculation has been used, for example, to normalize an assay for a target gene to an assay of an “endogenous control” gene and then to normalize the data to a calibrator sample such as an untreated reference sample to see if the treatment causes differential expression of the target gene. The equation has been typically applied to mRNA characterization and genomic DNA.

### BRIEF SUMMARY OF THE INVENTION

In one aspect, the present invention is directed to a method for preimplantation genetic diagnosis and fresh transfer of a day 3, day 4, day 5 or day 6 IVF embryo comprising (a) performing real-time PCR and 2^{-ΔΔC}_{T} analyses to determine normalized copy number of at least one invariant locus in the embryo on at least one chromosome of the IVF embryo; (b) determining the presence or absence of a genetic defect in the embryo based on the normalized copy number of the invariant loci in the embryo; and (c) transferring at least one embryo if determined to be without genetic defect within about 24 hours of performing step (a).

In one embodiment, the genetic defect is aneuploidy, e.g., nullisomy, monosomy, disomy, trisomy, and tetrasomy.

In another embodiment, the IVF embryo is also screened for a genetic defect that is not aneuploidy, e.g., a genetic defect selected from the group consisting of those provided in Table 2.

In yet another aspect, the invention is directed to a method for preimplantation genetic diagnosis of a day 3, day 4, day 5 or day 6 embyro, the diagnosis occurring within 24 hours prior to transfer of an embryo determined to be without genetic defect, the method comprising (a) performing realtime PCR and 2^{-ΔΔC}_{T} analyses to determine normalized copy number of at least one invariant locus in the embryo on at least one chromosome of the IVF embryo; and (b) determining the presence or absence of a genetic defect in the embryo based on the normalized copy number of the invariant loci in the embryo.

In one embodiment, the genetic defect is aneuploidy, e.g., nullisomy, monosomy, disomy, trisomy, and tetrasomy.

In another embodiment, the IVF embryo is also screened for a genetic defect that is not aneuploidy, e.g., a genetic defect selected from the group consisting of those provided in Table 2.

While the present invention permits PGD and the advantage of fresh transfer of an IVF embryo, it is also contemplated herein that steps (a) and (b) of the above methods may be performed and subsequently followed by freezing the embryo, including any embryo determined to be without genetic defect, e.g., if embryo transfer at a later date is more convenient or medically appropriate for the patient. It is also contemplated herein that steps (a) and (b) may be performed without a subsequent transfer step at all.

Thus, in a further aspect the present invention is directed to a method for preimplantation genetic diagnosis of a day 3, day 4, day 5 or day 6 IVF embryo comprising (a) performing real-time PCR and 2^{-ΔΔC}_{T} analyses to determine normalized copy number of at least one invariant locus in the embryo on at least one chromosome of the IVF embryo; (b) determining the presence or absence of a genetic defect in the embryo based on the normalized copy number of the invariant loci in the embryo; and (c) freezing said embryo.

In one embodiment, the genetic defect is aneuploidy, e.g., nullisomy, monosomy, disomy, trisomy, and tetrasomy.

In another embodiment, the IVF embryo is also screened for a genetic defect that is not aneuploidy, e.g., a genetic defect selected from the group consisting of those provided in Table 2.

In a further aspect, the invention is directed to a method for transferring an IVF embryo comprising (a) performing real-time PCR and 2^{-ΔΔC}_{T} analyses to determine the presence or absence of a genetic defect in the embryo based on normalized copy number of at least one invariant locus on at least one chromosome collected from at least one cell of the embryo; and (b) transferring the embryo if determined to be without genetic defect within about 154 hours of fertilization.

In one embodiment, the embryo is transferred between about 48 and about 144 hours of fertilization.

In further embodiments, the performing and transferring steps are accomplished within a period of about hours, about 24 hours, about 16 hours, about 12 hours, about 8 hours or about 5 hours.

In one embodiment, the genetic defect is aneuploidy, e.g., nullisomy, monosomy, disomy, trisomy, and tetrasomy.

In another embodiment, the IVF embryo is also screened for a genetic defect that is not aneuploidy, e.g., a genetic defect selected from the group consisting of those provided in Table 2.

In another aspect, the invention relates to a method for determining the presence or absence of a genetic defect in an IVF embryo prior to transfer comprising: (a) performing real-time PCR and 2^{-ΔΔC}_{T} analyses to determine normalized copy number of at least one invariant locus on at least one chromosome collected from at least one cell of the embryo and (b). selecting a candidate IVF embryo determined to be without genetic defect for transfer.

In one embodiment, the genetic defect is aneuploidy, e.g., nullisomy, monosomy, disomy, trisomy, and tetrasomy.

In another embodiment, the IVF embryo is also screened for a genetic defect that is not aneuploidy, e.g., a genetic defect selected from the group consisting of those provided in Table 2.

In another embodiment, determining the presence or absence of a genetic defect in the embryo comprises copy number analysis of at least one invariant locus on all of the chromosomes of the embryo.

In various additional embodiments, the IVF embryo is a human embryo, and may be a day 3, day 4, day 5 or day 6 embryo.

In a further embodiment, selecting a candidate IVF embryo determined to be without genetic defect is performed within 3-6 days of in vitro fertilization of said embryo.

In a further embodiment, the invariant loci are located on chromosomes selected from the group consisting of chromosomes 1, 2, 3, 4, 5, 6, 7, 8, 9, 10, 11, 12, 13, 14, 15, 16, 17, 18, 19, 20, 21 22, X and Y. In a particular embodiment, the chromosomes are chromosomes 13, 18, and 21.

In another embodiment, the method further comprises transferring the selected candidate IVF embryo on the same day as the steps of performing and selecting.

In an additional embodiment, the performing, selecting and transferring of the IVF embryo are accomplished within about 12 hours or less, within about 8 hours or less, or within about 5 hours or less.

In a particular embodiment, the IVF embryo is a blastocyst. In a further embodiment, the cells are biopsied from trophoectoderm.

In yet an additional embodiment, the performing, selecting and transferring of the blastocyst are accomplished within about 24 hours or less, within about 12 hours or less, within about 8 hours or less or within about 5 hours or less.

In yet additional embodiments, three or less IVF embryos are transferred, two or less IVF embryos are transferred, or one IVF embryo is transferred.

In an additional embodiment, determining the presence or absence of a genetic defect in the embryo is based on the copy number of about 100 or less invariant loci per chromosome, about 50 or less invariant loci per chromosome, about 40 or less invariant loci per chromosome, about 20 or less invariant loci per chromosome.

In another embodiment, determining the presence or absence of a genetic defect in the embryo is based on the copy number of at least two invariant loci, at least three invariant loci, at least five invariant loci, or at least ten invariant loci.

In a further aspect, the invention is directed to arrays comprising a plurality of nucleic acid probes comprising nucleic acid for at least one invariant locus from at least one human chromosome. In a particular embodiment, the probes are immobilized on a solid support. In an additional embodiment, the nucleic acid in the array comprises at least two invariant loci from at least one of human chromosomes 1-22, X and Y.

In another aspect, the invention relates to a method for making an array for preimplantation genetic diagnosis of an IVF embryo comprising (a) identifying at least one invariant loci for preimplantation genetic diagnosis, (b) selecting at least one invariant loci for at least one chromosome, and (c) affixing nucleic acid probes for the invariant loci on a solid support. In a particular embodiment, from about one to about 100 invariant loci for at least one chromosome are selected.

In a further embodiment, the invention is directed to kits comprising an array of nucleic acid probes immobilized on a solid support, the array comprising nucleic acid probes for at least one invariant locus from at least one human chromosome wherein the invariant loci are useful for determining the presence or absence of a genetic defect in an IVF embryo prior to transfer according to the methods of the invention.

### BRIEF DESCRIPTION OF THE DRAWINGS

FIG. 1 is a graph which provides an example of a real-time PCR amplification curve for a target locus (the human 18S ribosomal RNA gene, chromosome 22p12), in a normal female lymphocyte, (ID GM00321; Coriell Cell Repository, Camden, N.J.). Data for the curve was obtained by following the manufacturer's recommended protocol for real-time PCR (TAQMAN Gene Expression Assays Protocol Revision G and the TAQMAN Gene Expression Assay ID #Hs99999901 s1; Applied Biosystems (ABI), Foster City, Calif.). Data represents the cycle number at which a specific target sequence is amplified enough to reach an arbitrary threshold.

FIG. 2 is a plot illustrating the results of an analysis of chromosome 21 copy number using 5-cell lysates from 3 cell lines known to possess 1, 2 or 3 copies of chromosome 21 (chr21). DNA was collected from 8 samples (n=8) of 5 cells each from cell lines with the following karyotypes: 45, XY-21 (1 copy of chr21, Coriell ID GM01201); 46,XX (2 copies of chr21, Coriell ID GM00321); and 48,XY,+16,+21 (3 copies of chr21, Coriell ID GM04435). Target invariant loci included those indicated in Table 7 (FAM assays for chr21) from Applied Biosystems (ABI).

FIG. 3 is a plot illustrating the results of an analysis of chromosome X copy number using a 5 cell lysate from 4 cell lines known to possess 1, 2, 3 or 4 copies of chrX. DNA was collected from 8 samples (n=8) of 5 cells each from cell lines with the following karyotypes: 46,XY (1 copy of chrX, Coriell ID 00323); 46,XX (2 copies of chrX, Coriell ID GM00321); 47,XXX (3 copies of chrX, Coriell ID GM04626); and 49,XXXXY (4 copies chrX, Coriell ID GM00326). Target invariant loci included those indicated in Table 7 (FAM assays for chrX) from ABI.

FIG. 4 is a graph depicting the determination of chromosomal copy number in a trisomy 21 female (47,XX, +21, Coriell ID AG16777) according to the methods of the present invention. Four loci per chromosome (96 FAM assays found in Table 7) were evaluated according to the methods described in the present invention.

### DETAILED DESCRIPTION

While the specification concludes with the claims particularly pointing out and distinctly claiming the invention, it is believed that the present invention will be better understood from the following description.

All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Unless otherwise indicated, as used herein, “a” and “an” include the plural, such that, e.g., “a cell” can mean more than one cell.

As contemplated herein, Applicant's present invention is directed to methods for preimplantation genetic diagnosis of IVF embryos which uses RT-PCR methods to perform relative quantitation of chromosomal DNA copy number of invariant loci in single or few cells collected from an IVF embryo. Determination of chromosomal copy number in this way allows for the detection of aneuploidy in an IVF embryo.

Advantages associated with performing PGD according to the methods described herein include the ability to simultaneously characterize aneuploidy of all 24 chromosomes, single gene disorders, and other chromosomal abnormalities such as inheritance of reciprocal translocation derivatives. Significantly, the present method allows for an unprecedented ability to characterize the embryo for these abnormalities within 6 hours or less. This is a huge advance for the field of PGD because it provides the opportunity to perform analysis of a trophectoderm (TE) biopsy and select the embryo(s) for transfer in the same day. This avoids cryopreservation, may be less invasive than a blastomere biopsy since TE is extraembryonic, may give more accurate results than a day 3 blastomere biopsy since a TE biopsy yields more than a single cell, and may provide fewer sampling errors derived from mosaicism.

In addition, the short turn-around time also provides an opportunity to evaluate samples from around the world at one reference lab since delivery time can be accommodated and allow for fresh embryo transfer on day 5 after day 3 biopsy.

Furthermore, the present invention does not rely upon polymorphism characterization thereby allowing the same method to be applied to any IVF patients' embryos and without the need for parental DNA haplotype analysis. The method is also much more practical to perform since it is considerably less expensive than polymorphism microarray based methods previously described.

As contemplated herein, in order to determine chromosomal copy number of all chromosomes in a cell, for each chromosome, the threshold cycle (C_{T}) for at least one, but typically multiple, invariant loci found on the chromosome is determined. The average C_{T} for each chromosome is calculated and normalized by subtracting the average C_{T} determined for the other chromosomes (calculated by computing the combined average of all the C_{T} values obtained for all other chromosomes) from the average C_{T} determined for the chromosome. (See, Table 1, Equation 1). The process is repeated to determine the ΔC_{T} for each chromosome.

Multiple such C_{T} determinations are also performed on the corresponding chromosomes in a control, or reference, sample to obtain a ΔC_{T} for the chromosomes in the reference sample. As contemplated herein, reference samples are euploid cells (cells containing a normal number of chromosomes) and appropriate reference samples for comparison are familiar to one of skill in the art. For example, for assaying human IVF embryos, one could use 46,XY cells from a well characterized euploid cell line, e.g., cells from a repository such as Coriell that have a 46,XX (ID GM00323) or 46,XY (ID GM00321) karyotype.

Once ΔC_{T} values are obtained for a chromosome and its corresponding reference chromosome, subtraction of the ΔC_{T} of the reference chromosome from the ΔC_{T} of the corresponding chromosome is performed to generate a ΔΔC_{T} for each chromosome. (See, Table 1, Equation 2).

As understood by those of skill in the art, a 1 cycle difference is theoretically a 2 fold difference in quantity of the target DNA due to the exponential nature of PCR. Thus, the fold change of each chromosome relative to its corresponding reference sample may then be determined by applying Equation 3 in Table 1. In this calculation, ΔΔC_{T} determined for each chromosome serves as the negative exponent of 2 in order to calculate fold change for each chromosome relative to its corresponding reference sample.

Copy number of each chromosome may then be determined by multiplying the fold change by 2. (See, Table 1, Equation 4). As understood herein, a chromosomal specific fold change of 1 (i.e., a copy number of 2) indicates normal copy number of that chromosome in the IVF embryo. In contrast, a chromosome specific fold change of 0.5 or 1.5 would be considered abnormal, and indicate an IVF embryo with monosomy or trisomy of that chromosome, respectively. It is noted herein that this step is not performed for the Y chromosome, as normal males have one copy number.

TABLE 1
Determination of Chromosome Copy Number

Equation 1: Using C_{T} data collected for each
invariant loci, normalized value (ΔC_{T}) is
determined for each chromosome and a
corresponding normal reference chromosome
for at least one chromosome 1-22, X, and Y
(chromosome number is referred to
collectively herein as “N”).
ΔC_{T chN} = (average C_{T} for chromosome N assays) −
(average C_{T} for all other chromosome assays)
Equation 2: The ΔC_{T} for the reference sample
chromosome is subtracted from the test sample
ΔC_{T} of the same chromosome to generate a ΔΔC_{T}
for that chromosome.
ΔΔC_{T chN} = (test sample chromosome ΔC_{T chN}) −
(reference ΔC_{TchN})
Equation 3: The fold change of each chromosome
relative to the normal reference sample is
then calculated.
Fold Change_{chN} = 2^{-ΔΔCTchN}
Equation 4: Fold change is multiplied by 2 in
order to obtain a copy number for each
chromosome (not performed for Y chromosome and
not performed for X if reference sample is
male).
Copy Number = 2 × Fold Change_{chN}
Note:
Only autosomes are used to normalize the data for each individual chromosome N, where N is 1-22, X and Y.

As contemplated herein, copy number may be determined for any or all chromosomes in a cell according to the methods described herein. Specifically, it is understood that the methods may be employed to determine the copy number of all chromosomes, only a subset of chromosomes, or of a single chromosome of particular interest.

As contemplated herein, test sample data for chromosomes is normalized using only the average C_{T} determined for autosomes which ensures that the data is normalized using data for chromosomes with the best potential for a normal copy number of 2. Accordingly, it is understood that one would typically exclude data for the sex chromosomes from the calculation when subtracting the average C_{T} of “all other chromosomes” in Equation 1. In contrast to autosomes, which are present in pairs in euploid cells, as understood by one of skill in the art, the copy number of sex chromosomes can be normal and be either 0 (chrY in a normal female), 1 (chrX and Y in a normal male), or 2 copies (chrX in a normal female). As such, errors might be introduced if the data for a given chromosome was normalized with data including the sex chromosomes, since statistically approximately half the time the sample will be male (1 X and 1 Y) and half the time the sample will be female (2 X and 0 Y). It is also contemplated herein, however, that as the methods of the present invention can indicate the sex of an IVF embryo, it is possible that where the methods indicate that the X chromosome condition is disomy, X chromosome data could be included to normalize the chromosome data.

The methods described herein and detailed in Table 1 can be used to determine the copy number of sex chromosomes as well as autosomes. As contemplated herein, however, data for the sex chromosomes would be evaluated in a manner consistent with the gender of the reference sample used. For chrX, either a normal female or normal male could be used as the reference sample. If the reference sample is female, then the copy number can be calculated in a manner identical to that described for chr1-22 above. If the reference sample is male, than the fold change value wouldn't be multiplied by two and instead would already be equal to the copy number of chrX. For chrY, a normal male would need to be used as the reference sample and the fold change would be equal to the copy number and wouldn't require multiplication by 2.

In addition to monosomy or trisomy, other forms of aneuploidy, e.g., nullisomy, disomy (of the Y chromosome), and tetrasomy may also be detected according to the methods of the present invention. Chromosomal nullisomy may be diagnosed, for example, by detecting the absence of an amplification signal for a chromosome. Similarly, detecting 2 copies would indicate chromosomal disomy, and detecting 4 copies would reflect tetrasomy.

Data manipulation and calculations necessary to carry out the methods of the present invention may be performed according to any suitable means familiar to one of skill in the art. These include, but are not limited to commercially available computer software programs, e.g., Microsoft Excel, SDS (Applied Biosystems), or other similar computer software packages.

As contemplated herein, the methods of the present invention are useful for detecting the copy number of whole chromosomes. As used herein, a “whole” chromosome means that the intended resolution is a complete, intact chromosome, and not, e.g., a chromosomal fragment or microdeletion. Typically, if a microdeletion, balanced translocation or other similar chromosomal aberration occurs de novo in an embryo (i.e., such mutation isn't present in either parent and thus was not inherited by the embryo), it is unlikely to be diagnosed by the methods of the present invention. As such, it is contemplated herein that additional assays designed to detect such chromosomal defects in an IVF embryo may be performed in conjunction with the methods of the present invention.

In addition, where genetic disorders other than aneuploidy are known or suspected, it is contemplated herein that genetic assays to identify such abnormalities in the IVF embryo may be performed in concert with the methods of the present invention. Additional assays useful to detect such genetic disorders unrelated to copy number and applicable to PGD are familiar to one of skill in the art and include, for example, using sequencing primers capable of determining single gene disorder mutation sequences, or using primers designed for specific cytobands of a chromosome known to be on either side of known breakpoints in a patient with a reciprocal balanced translocation.

As used herein, the terms “locus” or “loci” (plural) refer to the position on a chromosome of a particular gene. The human genome is comprised of both “invariant” and “variable” loci. Variable loci are those loci for which alternative alleles exist and include, e.g., those alleles with single nucleotide polymorphisms (SNPS).

In contrast, as understood herein, “invariant loci” are positions in the genome of an organism for which no evidence exists of any polymorphism or variation in any population evaluated to date. As understood by one of skill in the art, invariant loci may typically be found in highly conserved regions encoding conserved functions in the human genome. These include, for example, the active sites of enzymes and the binding sites of protein receptors. Invariant loci are more likely found in exons; in contrast, one would expect to find a greater number of variable loci in introns or “junk DNA”.

According to the methods of the present invention, invariant loci are assayed to determine chromosomal copy number since analyzing loci possessing polymorphisms such as a SNP or a copy number variant (which may be found in normal “euploid” individuals) could indicate that the locus is present at a different copy number in different individuals with different polymorphisms. This could result in a misdiagnosis, e.g., of monosomy or trisomy, rather than indicating the true euploid nature of a cell. In addition, loci of variant nucleotide sequences can have different amplification efficiencies. For example, the efficiency of amplification of a SNP-containing locus may be less in one individual that is homozygous for an adenosine nucleotide (AA) than in another individual who is homozygous for a cytosine nucleotide (CC). As a result, the lower efficiency would make it appear as though the AA individual had fewer copies than the CC individual despite both individuals having two copies of the SNP-containing locus.

One of skill in the art may identify suitable invariant loci for use with the methods of the present invention by reviewing databases of genetic information. As used herein, the term “database of genetic information” includes databases which contain data characterizing the frequency of genetic loci for various populations and includes, for example, the Entrez SNP database available from NCBI, as well as databases available from NCI, WICGR, HGBASE or the International HapMap Project (see, e.g., International HapMap Consortium, Nature 449, 18 Oct. 2007, 851-862). Extensive proprietary SNP databases are also available through commercial vendors, and include, for example, Applied Biosystems' SNP database which supports their commercial TAQMAN SNP Genotyping Assays. Analyzing SNP databases would be useful with respect to the present invention, e.g., with regard to the identification of variant loci (i.e., loci with known polymorphisms) which could thus be avoided in the selection of loci for use as disclosed herein.

Other databases useful with respect to the methods of the present invention include databases which contain information regarding copy number variation. These databases are familiar to one of skill in the art and include, for example, Chromosome Abnormality Database (“CAD”; a collection of both constitutional and acquired abnormal karyotypes reported by UK regional cytogenetics centers); Database of Genomic Variants (a curated catalogue of large-scale variation in the human genome); DECIPHER (Database of Chromosomal Imbalance and Phenotype in Humans using Ensembl Resources, Decipher Consortium); ECARUCA (European Cytogeneticists Association Register of Unbalanced Chromosome Aberrations); Human Genomic Structural Variation Database (a catalogue of human genomic polymorphisms ascertained by experimental and computational analyses, Eichler laboratory, University of Washington, Seattle, Wash.); The Chromosome Anomaly Collection (contains examples of unbalanced chromosome abnormalities (UBCAs) without phenotypic effect; compiled by J. Barber, National Genetics Reference Laboratory (Wessex), Salisbury NHS Foundation Trust); CNV Project (The Copy Number Variation (CNV) Project Data Index; Sanger Institute); Structural Genome Variation.

Loci suitable for use in the methods of the present invention may also be identified by reviewing other publicly or commercially available genetic databases to identify loci that occur in a population at a frequency such that the loci may be deemed “invariant” and may be used to produce statistically useful data for PGD as contemplated herein. These databases include the Allele Frequency Database (“ALFRED”) supported by the US National Science Foundation. Based on the allele frequency data in this database, one could compile a list of loci to avoid using in the methods of the present invention; e.g., loci reported to exhibit different allele frequencies in different human populations. In addition, while not necessary to perform the methods of the present invention, based on the data provided in the ALFRED database, one could also identify and select suitable invariant loci for PGD in view of the parentage (e.g., ethnicity) of an IVF embryo to be analyzed.

Along with the review of genetic databases discussed above, factors for consideration in the selection of possible invariant loci for use with the methods of the invention include whether the locus sequence is specific (i.e. homologues or pseudogenes aren't present in the genome), whether primers and a probe can be designed to perform under common PCR conditions so that an assay for the locus can be performed under the same conditions as assays for other loci, and whether it is located in a region of the chromosome that is distant enough from other loci used so that, for example, both arms of each chromosome might be evaluated.

While it is contemplated herein that the loci deemed invariant in the Examples and Tables described herein may be used to screen for aneuploidy in any IVF embryo, it is possible that for any given invariant locus, there could exist a rare, previously undetected polymorphism in the human population. If an IVF embryo did possess such a rare, previously undetected polymorphism at a given “invariant” locus, chromosomal copy number could still be detected in that embryo where more than one locus per chromosome is employed in the methods provided herein.

Invariant loci may also be confirmed for use with a particular IVF embryo by using conventional methods to detect the presence of the invariant loci in the parental DNA using conventional methods. Analysis of parental DNA could be performed at any time, including prior to creation of the embryo.

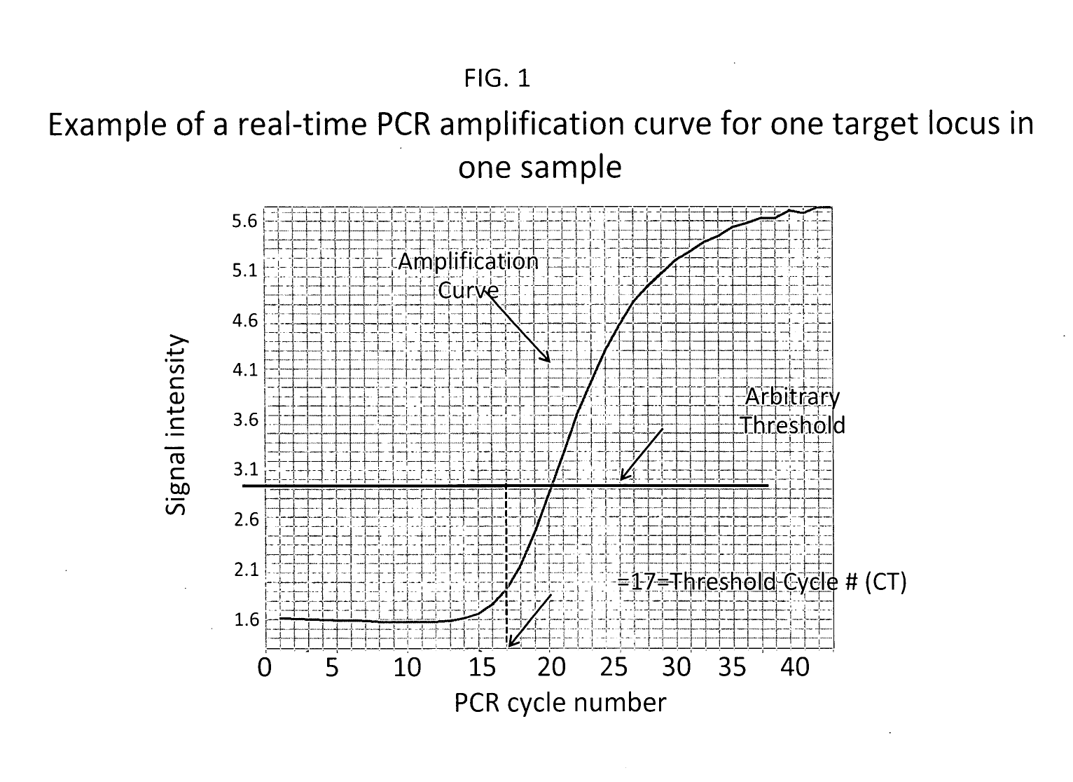

Once candidate invariant loci are identified in silico, one may then select any number for further analyses. Criteria for selection of various loci for additional testing include: whether an assay for the specific locus is readily available from a commercial supplier, whether an assay for the specific locus follows an expected sigmoidal pattern of amplification under standardized PCR conditions (i.e., see FIG. 1), and whether an assay for the locus performs well when evaluated on cells with known abnormalities. For example, suspected invariant loci may be utilized to screen euploid and aneuploid cells of known karyotype according to the methods of the present invention. In addition to using aneuploid and euploid cell lines, in some cases one may also biopsy frozen embryos, including previously diagnosed aneuploid embryos, to identify useful invariant loci to employ in the methods of the present invention. IVF embryos with karyotypes such as described in Table 4 are not uncommon; one of skill in the art, e.g., clinicians who routinely perform in vitro fertilization and PGD, typically have access to similar aneuploid embryos and have been given consent to use biopsied material from such embryos for research purposes.

By evaluating the ability of a particular locus to correctly identify copy number of a characterized sample, one of skill in the art can arrive at a set of invariant loci which can then be used to accurately predict the correct copy number for any given chromosome for PGD of an IVF embryo. As described in detail in the Examples and Tables provided herein below, such assays are easily performed and can be used to identify numerous suitable invariant loci for use in the practice of the present invention such as listed in detail in Table 7.

While any number of invariant loci may be used according to the methods of the present invention, suitable invariant loci may be identified and further characterized into sets and subsets of invariant loci for use as described herein. For example, in addition to displaying a lack of associated polymorphisms, and the ability to predict copy number of samples with known karyotype, subsets may be characterized based on various additional criteria including, but not limited to, the robustness of the chemistry and other technical factors associated with assaying a particular loci, e.g., availability of primers or other factors associated with amplifying particular loci, costs associated with use of particular loci and other practical aspects associated with performing the methods described herein that would be familiar to one of skill in the art.

Sets and/or subsets of invariant loci that may be used in the methods of the present invention may comprise hundreds of thousands of loci, but as contemplated herein, the methods of the present invention do not require the evaluation of a volume of data such as might be generated from a whole genome analysis. Rather, a set of invariant loci for use according to the methods of the present invention typically comprises less data than the entire genome of an individual, e.g., less than about 100,000, less than about 50,000, less than about 20,000, less than about 10,000, less than about 5,000, or less than about 1,000 invariant loci.

Loci may be evaluated for ability to predict chromosome copy number as provided herein and particular loci that perform well for each chromosome may be easily identified. As described above, invariant loci are more likely found in highly conserved regions in the genome, e.g., exons that code for proteins with key biochemical functions. Based on review of a publicly available human genome database, one of skill in the art can design primers that fall within a single exon and optimize such primers for performance in PCR and prediction of chromosome copy number as detailed herein. In addition, sets of assays that are designed to detect a single exon are commercially available. These “assays” or “sets of assays” include commercially available nucleic acid assays with fluorescently labeled probes typically used for gene expression analysis, e.g., ABI TAQMAN Gene Expression Assays. As contemplated herein, suitable commercial assays for use herein include those that are also capable of detecting genomic DNA.

As contemplated herein, any number of loci may be routinely selected for assay, however, since locus drop out from single cell amplification is likely, it is contemplated herein that typically at least more than one invariant locus would be evaluated per chromosome. For example, using 96 well plates and conventional high throughput methodologies, 192 loci (e.g., 96 samples in duplicate) may be easily assayed and provide accurate results according to the methods of the present invention.

By focusing on only invariant loci, a high quality set of loci may be obtained and used (in whole or in part in the form of subset(s) thereof) to analyze an embryo. Thus, by employing a robust set of invariant loci in combination with high throughput analysis using the comparative C_{T} method, it is contemplated herein that PGD may be performed more accurately, more efficiently and more rapidly than currently available modes of PGD analyses. For example, by employing the methods of the present invention, one may avoid the less efficient and error prone method of whole genome amplification based embryonic analysis which necessitates supplementary analysis of parental DNA to confirm embryonic data and/or to identify additional informative loci for embryonic genotyping.

As contemplated herein, the methods of the present invention permit assaying the embryo, determining the presence or absence of genetic defect, and selecting and transferring an embryo to be performed within a period of about 24 hours or less, e.g., within about 16, about 12, about 8 or about 5 hours or less. As such, same day cell biopsy and fresh transfer of an IVF embryo is possible. It is further contemplated that the method steps may be performed such than an embryo is determined to be without genetic defect and transferred within about 154 hours of fertilization, particularly between about 48 and about 144 hours of fertilization.

Although the methods of the present invention provide the advantage of PGD and fresh transfer, it is envisioned that there may be situations in which fresh transfer of an IVF embryo assayed and selected for transfer according to the methods of the present invention is not desired. Such situations may include, e.g., when fresh transfer is not convenient, or not medically appropriate. In such instances, it is contemplated herein that the embryo may be preserved, e.g., frozen or otherwise cryopreserved and maintained for possible transfer at a later date according to conventional methods. It is also possible that no embryo may be transferred.

As contemplated herein, nucleic acid probes for invariant loci can be provided in the form of an array for PGD according to the methods of the present invention. Such arrays are familiar to one of skill in the art and may be in various forms, including but not limited to, a solid support such as a chip or glass slide, e.g., in the form of a microarray or preloaded assay plate, to which the nucleic acid may be affixed or loaded according to methods familiar to one of skill in the art. Custom made assay chips with nucleic acid for loci of interest affixed or preloaded thereto may be obtained from commercial vendors, e.g., Applied Biosystems Inc. (Foster City, Calif.), Affymetrix Inc. (Santa Clara, Calif.), or Illumina Inc. (San Diego, Calif.).

Further contemplated herein are kits that comprise arrays for use with the methods of the present invention. For example, a kit might comprise an array of nucleic acid probes immobilized on a solid support or preloaded on an assay plate, the array comprising nucleic acid probes for at least one invariant locus from at least one human chromosome wherein the loci are informative for determining the presence or absence of a genetic defect in an IVF embryo prior to transfer according to the methods discussed herein. Additional components of such kits may comprise instructions as well as reagents, primers, probes or other tools of molecular biology familiar to one of skill in the art that might be of use in conducting PGD of an IVF embryo.

It is contemplated herein that PGD according to the methods of the present invention may be performed quite effectively utilizing the invariant loci provided herein. (See, e.g., Table 7). It is further contemplated, however, that additional invariant loci may be identified. As discussed above, such invariant loci may be identified in silico by reference to known databases of human genetic information, and may be further selected in view of data for a particular ethnic or geographic group. Selection of appropriate invariant loci may be confirmed by performing the methods of the present invention using control or reference samples of known karyotype and evaluating ability of the loci to correctly predict known copy number.

As envisioned, in order to maximize efficiency where possible and practical, data necessary to perform the methods of the present invention may be on hand when an IVF embryo becomes available for PGD. Thus, in order to facilitate the PGD and fresh transfer of an IVF embryo according to the methods of the present invention in the time frame described, invariant loci may be identified prior to the actual creation of an IVF embryo.

One of skill in the art will appreciate that the steps of the methods of the present invention may take place in different locations. For example, biological material may be obtained from the prospective parents of an IVF embryo and used to create an IVF embryo at the same clinic, or the materials may be transported according to conventional methods to a second location at which the IVF embryo may be created and maintained in vitro. Similarly, cells may be obtained from an IVF embryo and screened for PGD according to the methods of the present invention in the same laboratory, or the cells may be delivered to a second location for PGD. If PGD is performed at a different location than where the embryo is maintained, PGD results may be transmitted back to the laboratory maintaining the embryo where transfer of suitable embryos into the recipient may then be performed. As would be apparent to one of skill in the art, the steps of the method are ideally performed in locations such that the entire process takes place in the most efficient manner possible; for example, in one embodiment, the IVF embryo is maintained in the same facility in which the genetic screening is performed and in which embryo transfer takes place. In this way, loss of time associated with shipping the cells to a second laboratory for genetic analysis is avoided. This would be especially advantageous where the time available for performing PGD and embryo transfer is extremely limited, for example, with regard to the same day biopsy, PGD and fresh transfer of a day 5 or day 6 blastocyst.

As contemplated herein, genetic analysis of an “embryo” includes assay of nucleic acid from cells from an IVF embryo (an embryo fertilized not less than about 40 hours before analysis), cells from a blastocyst (typically an embryo at day 4, day 5 or day 6 after fertilization) as well as cells biopsied from an embryo but of extraembryonic origin, e.g., trophectoderm, or polar bodies. The plural form of this term is included, such that, the term “an embryo” as used herein contemplates that more than one embryo or blastocyst may be concurrently assayed or transferred according to the methods of the present invention.

It is further contemplated herein that more than one cell of an embryo may be biopsied as conditions permit, for example, at least one cell of trophectoderm may be biopsied and assayed according to the methods of the present invention. Assaying more than one cell in this way can be used to detect mosaicism in an embryo (a condition in which cells in an embryo may differ genetically from other cells in the embryo) which cannot be detected if only a single cell is assayed. Thus, as contemplated herein, the methods of the present invention can be used to biopsy a day 5 or day 6 embryo, screen the embryo for mosaicism, and still permit fresh transfer of the embryo on the same day.

“Transferring” an IVF embryo refers to the process of placing an IVF embryo into a female patient with the objective that the embryo will implant and result in a viable pregnancy.

The term “fresh transfer” refers to the transfer of an embryo which has not been subjected to cryogenic preservation.

As used herein, a “plurality of chromosomes” refers to more than one chromosome.

“Candidate IVF embryos” are those embryos determined to be without genetic defect according to the methods of the present invention. These embryos may be deemed suitable for transfer, however, it is understood that other criteria familiar to one of skill in the art may also be taken into consideration in the selection of particular embryos for transfer.

The terms “chromosomal abnormality” and “genetic defect” are used interchangeably herein and refer to a deviation between the structure or copy number of the subject chromosome and a normal chromosome. The term “normal” refers to the predominate karyotype or banding pattern found in healthy individuals of a particular species. A chromosomal abnormality or genetic defect can be numerical or structural, and includes but is not limited to, single gene defects, sex-linked disorders, or chromosomal disorders, e.g., aneuploidy, polyploidy, inversion, a trisomy, a monosomy, duplication, deletion or additions of entire chromosomes or parts thereof, insertions, rearrangements, and translocations. As provided herein, the methods of the present invention may be used to detect aneuploidy in an IVF embryo, however, it is further contemplated herein that an IVF embryo may be screened for additional chromosomal abnormalities other than aneuploidy. Additional genetic screening may be performed using methods familiar to one of skill in the art and concurrently with the methods of the present invention.

Chromosomal abnormalities or genetic defects can be correlated with presence of a pathological condition or with a predisposition to develop a pathological condition. Numerous examples of pathological conditions associated with genetic defects on particular chromosomes and/or linked to a particular gene are known to those of skill in the art and literature references and electronic databases containing extensive and detailed information describing genetic defects are widely available. With such knowledge, one may employ the methods of the present invention alone or in combination with additional diagnostic methods to determine copy number and/or the presence of other chromosomal abnormalities in order to elucidate whether an IVF embryo may possess or be prone to a particular pathological condition. Such conditions include, for example, the genetic diseases listed herein in Table 2 and as described in U.S. Pat. No. 7,439,346.

TABLE 2
Examples of Genetic Disease

| Chromosome | |
|---|---|
| Number | Genetic Disease |
| 13 | Breast and ovarian cancers, deafness, |
| | Wilson's Disease. Patau's Syndrome |
| 15 | Marfan Syndrome, Tay-Sach's Disease |
| 16 | Polycystic kidney disease, Alpha thalassemia |
| 17 | Charcot-Marie-Tooth Disease |
| 18 | Niemann-Pick Disease, pancreatic cancer, |
| | Edward's Syndrome |
| 21 | Down's Syndrome |
| X | Duchenne muscular dystrophy (DMD), Turner's |
| | Syndrome, Fragile X Syndrome, Klinefelter's |
| | Syndrome |
| | X-linked diseases: hemophilia, |
| | adrenoleukodystrophy, and Hunter's disease |
| Y | Acute myeloid leukemia |

Specific examples of pathological conditions associated with aneuploidy which may be detected in an IVF embryo according to the methods of the present invention include: Turner's Syndrome (a single X chromosome, e.g., 45, X or 45, X0); Klinefelter's Syndrome (an extra X chromosome, e.g., 47, XXY); Edward's Syndrome (three copies of chromosome 18); Down's Syndrome (three copies of chromosome 21); Patau's Syndrome (three copies of chromosome 13), trisomy 8, trisomy 9 and trisomy 16.

As discussed above, in addition to screening an IVF embryo for aneuploidy according to the methods of the present invention, it is further contemplated herein that an IVF embryo may also be screened for other genetic disorders using additional methods familiar to one of skill in the art. For example, an embryo may be concurrently screened for chromosomal microdeletions, translocations, or rearrangements using targeted preamplification strategies performed in parallel with the methods of the present invention. As used herein, a “targeted preamplification strategy” could include creation of custom probes for specific patients in order to successfully screen for inheritable disorders. Such additional screening methods can include for example, single nucleotide polymorphism real-time PCR, sequencing, restriction fragment polymorphism analysis, or short tandem repeat fragment size analyses. Such methods are familiar to one of skill in the art.

In addition, as mentioned herein above an IVF embryo may be screened for genetic defects according to the methods of the present invention while also being screened for at least one single gene genetic disorder. Such disorders may be detected based on the presence or absence of a SNP allele identified according to conventional methods or as described in copending US patent application U.S. Ser. No. 61/205,522. For example, if a single gene disorder were identified in the parents of an IVF embryo, that disorder could be evaluated in conjunction with the methods of the present invention by co-amplifying the disease causing target sequence or linked DNA sequences with the (aneuploidy informative) invariant loci. Analysis of the aneuploidy state of each chromosome and the presence or absence of the single gene disorder could then be evaluated in each resulting embryo.

As used herein, an IVF embryo “determined to be without genetic defect” refers to an embryo that is determined to be free of a particular genetic defect for which it was screened. It is understood that while the methods of the present invention are accurate, they may not be able to detect 100% of the genetic abnormalities in an IVF embryo. In some cases, data may be interpreted as meaning that there is a greatly reduced chance of the IVF embryo having a particular genetic defect, e.g., as would be the case for diagnosing mosaicism in an embryo, given that only a few cells, at best, may be assayed.

Methods for PGD described herein involve nucleic acid analysis; standard techniques for nucleic acid isolation and purification are known and are described in, for example, in Miller (ed.) 1972 Experiments in Molecular Genetics, Cold Spring Harbor Laboratory, Cold Spring Harbor, N.Y.; Old and Primrose, 1994 Principles of Gene Manipulation, 5th ed., University of California Press, Berkeley; Schleif and Wensink, 1982 Practical Methods in Molecular Biology; Glover (Ed.) 1985 DNA Cloning: Vols. I AND II, IRL Press, Oxford, UK; Harnes and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York City.

The sequence of a nucleic acid may be determined as necessary using conventional methods. These methods include, for example, PCR, gel electrophoresis, ELISA, mass spectrometry, MALDI-TOF mass spectrometry hybridization, primer extension, fluorescence detection, fluorescence resonance energy transfer (FRET), fluorescence polarization, DNA sequencing, Sanger dideoxy sequencing, DNA sequencing gels, capillary electrophoresis on an automated DNA sequencing machine, microchannel electrophoresis, microarray, southern blot, slot blot, dot blot, single primer linear nucleic acid amplification, as described in U.S. Pat. No. 6,251,639, SNP-IT, GeneChips®, HuSNP®, BeadArray, TAQMAN® assay, Invader® assay, MassEXTEND®, or MassCLEAVE™ (hMC) method.

Nucleic acid amplification methods are also known and may be used in PGD as contemplated herein, including the polymerase chain reaction (PCR) (PCR Protocols, A Guide to Methods and Applications, ed. Innis, Academic Press, N.Y. 1990; PCR: A Practical Approach, M. J. McPherson, et al., IRL Press (1991)) and particularly real-time, quantitative PCR discussed hereinabove (Schmittgen, T. and Livak, K., Nature Protocols 2008; Vol 3, No. 6:1101-1108; Livak, K. and Schmittgen, T., Methods 2001; 25:402-408). Other known amplification methods include ligase chain reaction (LCR) (Landegren, Science 1988; 241:1077); transcription amplification (Kwoh, Proc. Natl. Acad. Sci. USA 1989; 86:1173); self-sustained sequence replication (Guatelli, Proc. Natl. Acad. Sci. USA 1990; 87:1874); Q Beta replicase amplification (Smith, J., Clin. Microbial. 1997; 35:1477-1491), and other RNA polymerase mediated techniques such as nucleic acid sequence based amplification, NASBA (U.S. Pat. Nos. 4,683,195 and 4,683,202); 3SR (self-sustained sequence reaction); RACE-PCR (rapid amplification of cDNA ends); PLCR (a combination of polymerase chain reaction and ligase chain reaction); SDA (strand displacement amplification); and SOE-PCR (splice overlap extension PCR).

Errors associated with amplification include allelic, or locus dropout (LDO) and such errors are familiar to one of skill in the art. LDO rate estimates can differ based on the method by which they are measured. For example, microarrays often underestimate LDO rates. There are analysis settings that can be adjusted that will lead to different estimates, e.g., stringent genotype calls are associated with higher LDO rate estimates. Real time PCR is the most stringent method for evaluating LDO so estimates based on the use of real time PCR may be higher than microarray measures.

In order to avoid misdiagnoses due to amplification errors such as allelic or locus drop out, it is understood herein that sequencing an allele of interest may include sequencing nucleic acid around the allele to ensure amplification accuracy. For example, the disease-causative allele may be physically linked (close together) with a non-causitive allele nearby in the DNA sequence. These two sites in the DNA are very likely to be inherited together, barring any meiotic recombination between the sites. Sites nearer each other are less likely to undergo recombination. As a result, the non-causative allele can be used as a confirmatory marker of the disease causing allele in order to avoid misdiagnosis from disease allele PCR dropout. Such techniques are familiar to one of skill in the art.

It is contemplated herein that conventional methods to analyze nucleic acid include methods that permit the analysis of nucleic acid from a small number of cells. Such methods may include performing a “preamplification” of DNA prior to real-time PCR using invariant locus specific primers. Such methods are a modification of methods familiar to one of skill in the art, and kits to perform such preamplification are commercially available, for example, TAQMAN® PreAmp Cells-to-Ct™ Kit from Applied Biosystems. While these kits are designed to preamplify cDNA derived from RNA, they can also be used successfully on genomic DNA.

As contemplated herein, in a particular embodiment the methods of the present invention are performed using means which permit multiple, parallel real-time PCR reactions, including, but not limited to, high throughput genotyping using one or more assay platforms. By evaluating multiple loci of interest in this way, preimplantation genetic diagnosis may be performed quickly and efficiently such that embryo diagnosis and transfer may occur without the need for cryopreservation of the embryo; ideally, such steps occur in the same day. For example, it is contemplated herein that candidate embryo selection and embryo transfer may be performed within about 5 hours after biopsy of the embryo for diagnosis. It is further contemplated herein that the methods of the present invention may permit the genetic screening of all chromosomes of an IVF embryo followed by fresh transfer of the embryo on the same day.

As discussed above, materials and methods for high throughput real-time PCR, including the basic concept of analyzing real-time PCR data by the comparative C_{T} method, are familiar to those of skill in the art. These include, but are not limited to, commercial real-time PCR assay plates designed for such purposes. For example, high throughput methodologies which may be employed to practice the methods of the present invention include commercially available microarray plates that use nanoliter fluidics, for example, Applied Biosystems' TAQMAN OpenArray™ Genotyping Plates, which may be customized to contain nucleic acid for invariant loci of interest. Additional systems include the Fluidigm Inc. BioMark System for genetic analysis, or the Roche Applied Science LightCycler 1536 Real-Time PCR System.

Suitable primers for use in the methods of the present invention may be obtained from commercial vendors. They may also be designed by one of skill in the art according to conventional methods and published sequence information for any loci of interest.

Each loci-specific PCR reaction may include probes with unique fluorescent properties and reaction products will result in a different wavelength of quantifiable fluorescence. For example, gene expression assays which include a probe with a FAM label are commercially available (e.g., TAQMAN gene expression assays, Applied Biosystems). Assays can also be designed to include a VIC label on the probe. The loci of interest can be analyzed using conventional gel electrophoresis followed by fluorescence detection or read using a commercially available fluorescent plate reader or scanner. Commercially available computer imaging systems designed for high throughput include, e.g., 7900HT (Applied Biosystems Inc.), BioMark (Fluidigm, Inc.), or the LightCycler® 480 (Roche). The resulting fluorescence data can be evaluated with statistical protocols and computer programs such as SDS (Applied Biosystems), or Microsoft Excel (Microsoft Inc.). (See, e.g., Livak, K. L. and Todd, J. A., Nature Genetics Vol. 9, April 1995, 341-342).

The methods of the present invention may be used to screen at least one candidate IVF embryo concurrently such that more than one IVF embryo without genetic defect may be identified and transferred. The number of such embryos that may be appropriate to transfer may be determined by one of skill in the art according to conventional methods.

All publications cited in the specification are indicative of the level of skill of those skilled in the art to which this invention pertains. All these publications are herein incorporated by reference in their entirety to the same extent as if each individual publication were specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Examples

### Example 1

### Identification of Invariant Loci and Analysis of Chromosome 13 Copy Number in Cell Lines and Embryonic Tissue using Real-Time PCR and 2^{-ΔΔC}_{T} Analyses

Genetic loci suitable for predicting chromosomal copy number according to the methods of the present invention (”invariant loci“) were identified by assaying the ability of loci to accurately predict the correct chromosomal copy number of cells of known karyotype (Coriell Cell Repository, Camden, NJ) (Table 3) and cells from frozen aneuploid embryos (Table 4).

In this example we established a set of loci for chromosome 13 that perform well. To do this, we obtained cells from cell lines and embryos that were previously determined to be either 46,XX (GM00321, Table 3), 47,XY,+13 (GMO2948, Table 3), or 45,XY,−13 (embryo 8, Table 4).

For the analysis of copy number of chromosome 13 in the given samples, 8 invariant loci (as FAM labeled assays) were selected based on commercial availability and design specific for exon detection (s1 assay designation, ABI) and are listed in Table 5. Targeting loci within an exon will provide the most opportunity for avoiding areas of the genome with polymorphisms (genetic variation in normal euploid cells). This is due to the fact that exon sequences are more critical to protein function than are other regions such as introns or “junk DNA.” For that reason, exon sequences are more highly evolutionarily conserved and less variable, making them the ideal target for this method. In addition, exon sequences are well characterized for possible polymorphisms that can be evaluated in silico by referring to the aforementioned genetic databases.

In this experiment, embryo biopsy and cell line lysates were preamplified with 16 cycles. Preamplified DNA was then loaded in quadruplicate into a Fluidigm BioMark RealTime PCR 96.96 Gene Expression Array according to the manufacturer's instructions (Fludigm Inc.) and using Gene Expression Master Mix (ABI). C_{T} values were obtained for each assay for each replicate for each sample.

In this case, instead of computing the average C_{T} for all chromosome 13 loci, each individual locus for chromosome 13 was first computed separately to generate a copy number prediction. The copy number prediction of each locus was then evaluated for accuracy against the reference data for these samples. The 4 loci with the fewest errors (indicated in bold in Table 5) were identified. These loci provided the best accuracy of copy number assignments considering what to expect based on previously determining the karyotypes of the samples used. Once identified, the 4 best performing loci were then reanalyzed as an average C_{T} to determine the copy number for chromosome 13 (Table 6). This provided confirmation that these 4 loci were sufficient to provide an accurate diagnosis (i.e., loci associated with chromosome 13 that can reliably identify the correct copy number). Moreover, these data indicate that these 4 loci are acceptable to use for aneuploidy diagnosis of chromosome 13 in PGD of any IVF embryo.

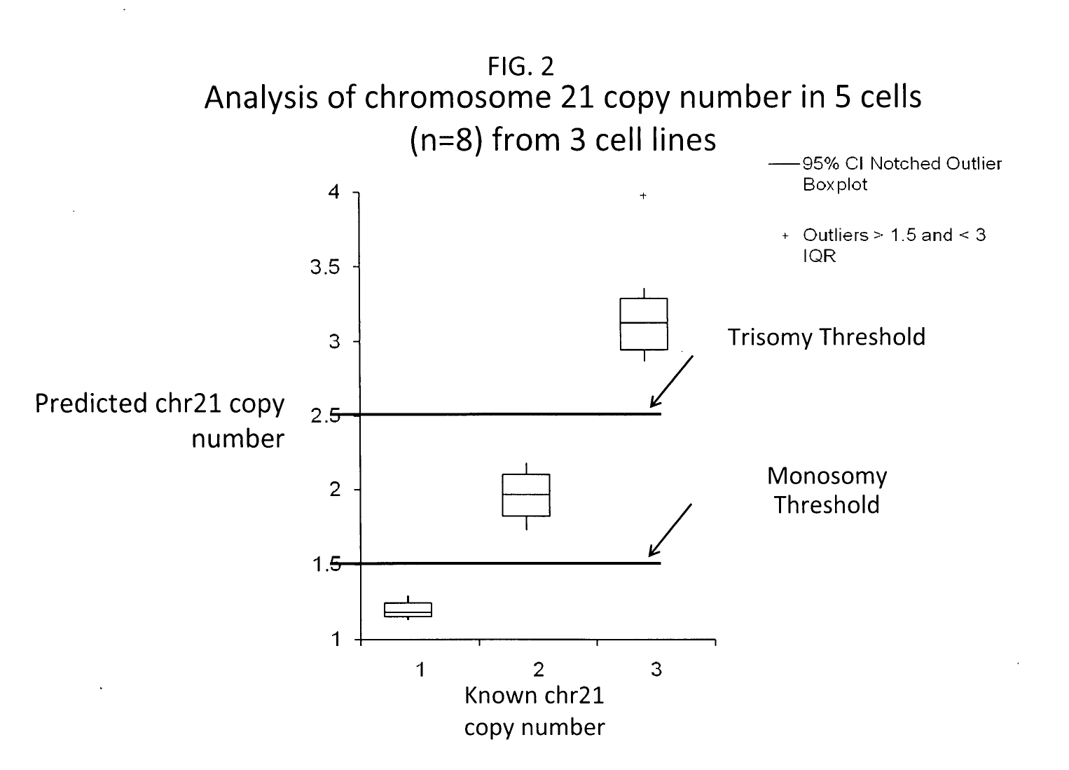

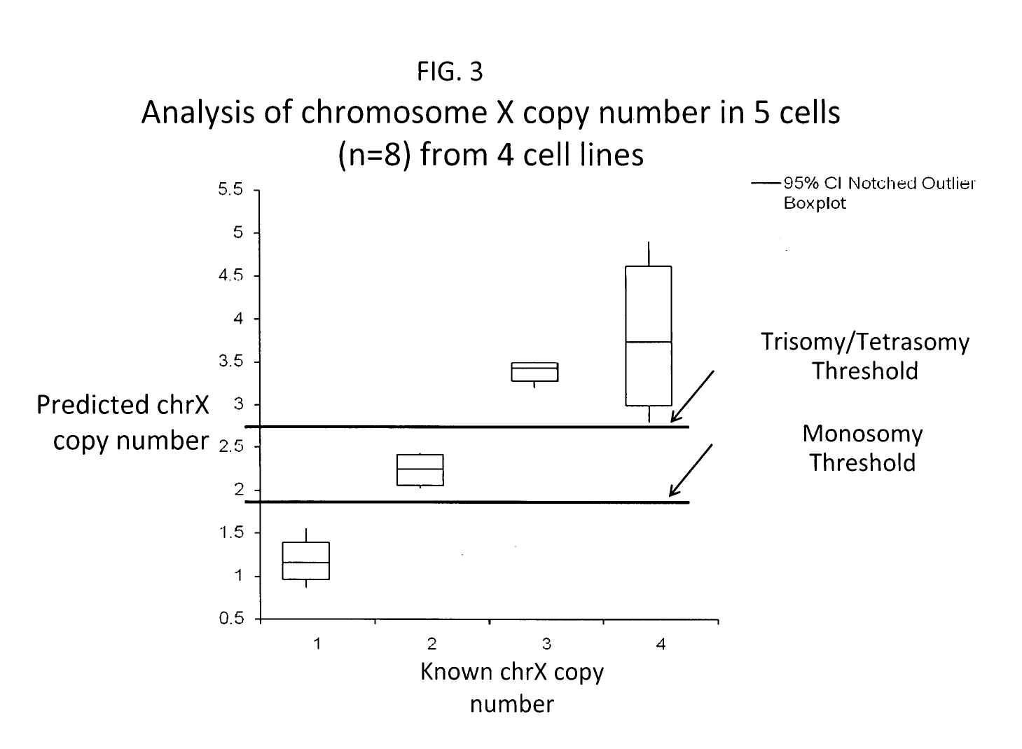

This process was repeated in order to obtain reference data for all 24 chromosomes to establish 192 loci (Table 7) which may be reliably used to indicate correct chromosomal copy number and using combinations of cell lines and embryos described in Table 3 and 4. To further illustrate the criteria for identification of useful loci, examples of the separation observed for chromosome X and 21 are shown in FIGS. 2 and 3 and further described below.

TABLE 3
Cell Lines

| Coriell Cell | |
|---|---|
| Repository ID | Karyotype |
| GM00321 | 46, XX |
| GM09286 | 47, XY, +9 |
| GM02948 | 47, XY, +13 |
| GM01359 | 47, XY, +18 |
| GM04610 | 47, XX, +8 |
| GM03184 | 47, XY, +15 |
| GM04435 | 48, XY,, +16, +21 |
| GM04626 | 47, XXX |
| GM01201 | 45, XX, −21 |
| GM00326 | 49, XXXXY |
| GM02067 | 47, XY, +21 |
| GM00323 | 46, XY |
| GM07106 | 47, XY, +22s+ |
| GM00875 | 45, XO |
| GM00325 | 47, XXY |
| GM09326 | 47, XYY |
| GM02521 | 48, XXYY |
| GM11420 | 49, XYYYY |
| GM11534 | 56, XY, t(11; 22)(q24; |
| | q12), +5, +7, +8, +15, +18, +20, +21, +der(22)t(11; |
| | 22)(q24; q12) + mars |
| GM10401 | 47, XX, +2 |
| GM01454 | 47, XY, +12 |
| GM07408 | 47, XX, +20 |
| GM00980 | 45, XX, +der(11), −22 |

TABLE 4
Embryos

| Embryo | |
|---|---|
| Number | Karyotype |
| 1 | 46, XO, +3, +16, −19 |
| 2 | 44, XY, −11, −13 |
| 3 | 46, XX, +8, −22 |
| 4 | 44, XX, −10, −16 |
| 5 | 46, XY, +der(5)t(3; 5)(q26.2; |
| | p15.1), −7, +11, −15, −16, +17, −18, +22 |
| 6 | 46, XX, der(5)t(5: 17)(q13; q21.3), +9, −22 |
| 7 | 45, XX, −19 |
| 8 | 45, XY, −13 |
| 9 | 48, XXX, +12 |
| 10 | 45, XY, −8 |
| 11 | 45, XY, −4 |
| 12 | 46, XX, −15, +17 |
| 13 | 47, XY, +16 |
| 14 | 44, XX, −1, −4, +16, −22 |
| 15 | 44, XY, −9, −14 |
| 16 | 45, XY, −6 |
| 17 | 48, XY, +1, +12 |
| 18 | 47, XX, +14 |
| 19 | 47, XY, +15 |
| 20 | 48, XY, +17, +22 |
| 21 | 47, XX, +22 |
| 22 | 45, XX, −22 |
| 23 | 48, XX, +13, +15, −21, +22 |
| 24 | 47, XY, +22 |
| 25 | 45, XY, −22 |
| 26 | 45, XX, −12 |
| 27 | 45, XX, −13 |
| 28 | 47, XXY, −7, +14 |
| 29 | 45, XY, −4 |
| 30 | 44, XY, −20, −21 |
| 31 | 47, XY, +6 |
| 32 | 45, XY, −17 |
| 33 | 43, XY, −7, −13, −14 |
| 34 | 47, XX, +18 |
| 35 | 45, XX, −8 |
| 36 | 45, XY, −22 |
| 37 | 45, XX, −15 |
| 38 | 48, XY, +5, +16 |
| 39 | 47, XX, +der(2)t(2: 20)(q21; p12.2), +17, −20 |
| 40 | 47, XX, +7 |
| 41 | 47, XY, +9, +19, −21 |
| 42 | 45, XX, −17 |
| 43 | 48, XXX, +2, +13, −19 |
| 44 | 45, XX, −2 |
| 45 | 44, XY, −5, −11 |

TABLE 5
Chromosome 13 assays tested

| Assay ID | Gene Symbol | Gene Name | Chromosome |
|---|---|---|---|
| Hs00937168_{—}s1 | CYSLTR2 | cysteinyl leukotriene receptor 2 | 13 q 14.2 |
| Hs01028557_s1 | SLITRK1 | SLIT and NTRK-like family, member 1 | 13 q 31.1 |
| Hs01037385_s1 | HMGB1 | high-mobility group box 1 | 13 q 12.3 |
| Hs01072517_{—}s1 | SOX21 | SRY (sex determining region Y)-box 21 | 13 q 32.1 |
| Hs01635854_{—}s1 | SIAH3 | seven in absentia homolog 3 (Drosophila) | 13 q 14.12 |
| Hs01650625_s1 | KBTBD6 | kelch repeat and BTB (POZ) domain | 13 q 14.11 |
| | | containing 6 | |
| Hs01879077_{—}s1 | UTP14C | UTP14, U3 small nucleolar | 13 q 14.3 |
| | | ribonucleoprotein, homolog C (yeast) | |
| HS01921463_s1 | GPR18 | G protein-coupled receptor 18 | 13 q 32.3 |

Assays indicated in bold performed well on cells with known abnormality for chr13.

TABLE 6
Copy number results on chromosome 13 using the best performing assays from Table 5.

| | Sample Type | | | | | | | | |
|---|---|---|---|---|---|---|---|---|---|
| | Embryo 8 | GM00321 | GM00321 | GM00321 | GM00321 | GM02948 | GM02948 | GM02948 | GM02948 |
| Karyotype | 45, XY, −13 | 46, XX | 46, XX | 46, XX | 46, XX | 47, XY, +13 | 47, XY, +13 | 47, XY, +13 | 47, XY, +13 |
| Copy Number | 1.0 | 1.9 | 2.1 | 2.1 | 1.9 | 2.8 | 3.4 | 3.7 | 3.4 |

For chromosome 21 (chr21), DNA was collected from eight 5-cell samples from cell lines with the following karyotypes: either 45, XY-21 (1 copy of chr21, Coriell ID GM01201); 46,XX (2 copies of chr21, Coriell ID GM00321); or 48,XY,+16,+21 (3 copies of chr21, Coriell ID GM04435).

The results provided in FIG. 2 show box and mean plots and summary statistics for the samples using the best performing loci indicated in Table 7. The best performing loci were determined using methods similar to those described above for chromosome 13. Statistics include the number of observations analyzed, and the mean, median, standard deviation, standard error, minimum, maximum and interquartile range (IQR). Confidence intervals are calculated for the mean and median. The interval shows, for the 95% level of certainty, the range of the true underlying population mean. Results indicate that copy number of chromosome 21 was accurately detected by assaying the 4 loci (indicated in Table 7) according to the methods of the present invention.

Importantly, as seen in FIG. 2, the results indicate that the distributions of observed copy number values for abnormal cells with 1 or 3 copies of chr21 do not overlap with the distribution of data for the euploid cells with 2 copies of chr21. Thus, a “threshold” copy number value may be established based on these data. In this context, a “threshold” refers to a copy number value such that different copy number values above and below the threshold may be assigned with confidence. For example, a monosomy copy number threshold could be applied to this data so that cells with monosomy (1 copy) are always below and cells with disomy (2 copies) are always above the threshold. Similarly, a trisomy copy number threshold could be applied to this data so that cells with trisomy are always above and cells with disomy are always below a given threshold. Possible thresholds meeting this description are indicated with a black bar in FIG. 2. Previously defined thresholds for chromosome 21, provide the opportunity to test an embryo and assign a copy number with a high degree of sensitivity and specificity.

For chromosome X (chrX), similar predictive copy number values and aneuploidy thresholds for analysis were obtained. Specifically, chromosome X copy number was analyzed according to the methods of the present invention using eight 5-cell lysates from each of 4 cell lines known to possess 1, 2, 3 or 4 copies of chrX. Specifically, DNA was collected from cell lines with the following karyotypes: 46,XY (1 chrX copy, Coriell ID 00323); 46,XX (2 chrX copies, Coriell ID GM00321); 47,XXX (3 chrX copies, Coriell ID GM04626); and 49,XXXXY (4 chrX copies, Coriell ID GM00326). The best performing chrX loci (ChrX FAM assays) were determined as described for chromosome 13 and are provided in Table 7. The data for these best performing loci are represented in the box plot in FIG. 3 with possible thresholds indicated with black bars. Results indicate that, using the indicated loci, the methods of the present invention were able to accurately predict the copy number of chrX in the tested reference samples.

TABLE 7
Examples of useful assays defined by analysis of cell
lines and embryos.

| VIC Assay ID | Gene Symbol | Chromosome | FAM Assay ID | Gene Symbol | Chromosome |
|---|---|---|---|---|---|
| Hs00920816_s1 | GJB4 | 1 | Hs00329637_s1 | KIAA1026 | 1 |
| Hs00921604_s1 | OR6K3 | 1 | Hs00539900_s1 | C1orf116 | 1 |
| Hs00947222_s1 | LOC148696 | 1 | Hs00541426_s1 | C1orf65 | 1 |
| Hs00954595_s1 | SPRR1A | 1 | Hs01104142_s1 | C1orf68 | 1 |
| Hs00540269_s1 | RHBDD1 | 2 | Hs00703942_s1 | C2orf53 | 2 |
| Hs00983296_s1 | LOC1720 | 2 | Hs00706886_s1 | C2orf83 | 2 |
| Hs01372895_s1 | OR6B3 | 2 | Hs00707637_s1 | C2orf16 | 2 |
| Hs01374521_s1 | UGT1A5 | 2 | Hs00745096_s1 | C2orf19 | 2 |
| Hs00928897_s1 | CCR1 | 3 | Hs00261464_s1 | C3orf42 | 3 |
| Hs01053049_s1 | SOX2 | 3 | Hs00748167_s1 | C3orf48 | 3 |
| Hs01053201_s1 | ZBTB38 | 3 | Hs01907876_s1 | CCBP2 | 3 |
| Hs01868189_s1 | TRIM59 | 3 | Hs01930174_s1 | OR5AC2 | 3 |
| Hs00944192_s1 | ATOH1 | 4 | Hs00257530_s1 | C4orf23 | 4 |
| Hs01043024_s1 | PDHA2 | 4 | Hs00259260_s1 | C4orf18 | 4 |
| Hs01872448_s1 | TLR2 | 4 | Hs00539499_s1 | C4orf30 | 4 |
| Hs01877256_s1 | RHOH | 4 | Hs00758583_s1 | C4orf39 | 4 |
| Hs00746872_s1 | NBPF22P | 5 | Hs00535083_s1 | C5orf4 | 5 |
| Hs00972208_s1 | GPR151 | 5 | Hs00924759_s1 | PCDHB9 | 5 |
| Hs01585827_s1 | PCDHB10 | 5 | Hs00950829_s1 | OR2V2 | 5 |
| Hs01594572_s1 | PCDHA7 | 5 | Hs01588662_s1 | C5orf39 | 5 |
| Hs00745107_s1 | HLA-DQB2 | 6 | Hs00256056_s1 | C6orf106 | 6 |
| Hs01038522_s1 | CNR1 | 6 | Hs00832315_s1 | C6orf66 | 6 |
| Hs01065322_s1 | PAQR8 | 6 | Hs00918806_s1 | OR5V1 | 6 |
| Hs01894157_s1 | OR2H1 | 6 | Hs00937283_s1 | OR2B3 | 6 |
| Hs00541664_s1 | DKFZP586I1420 | 7 | Hs00290888_s1 | C7orf41 | 7 |
| Hs01073352_s1 | OR2A2 | 7 | Hs00760712_s1 | MKRN1 | 7 |
| Hs01114065_s1 | OR2A5 | 7 | Hs00976831_s1 | CLDN4 | 7 |
| Hs01114862_s1 | OR2A12 | 7 | Hs01010356_s1 | OR9A2 | 7 |
| Hs00535362_s1 | ASAP1 | 8 | Hs00252427_s1 | C8orf17 | 8 |
| Hs00976551_s1 | NPBWR1 | 8 | Hs00535539_s1 | C8orf4 | 8 |
| Hs01084964_s1 | BHLHE22 | 8 | Hs00703902_s1 | C8orf77 | 8 |
| Hs01854954_s1 | NAT2 | 8 | Hs00708650_s1 | C8orf15 | 8 |
| Hs00762234_s1 | SSNA1 | 9 | Hs00275297_s1 | C9orf53 | 9 |
| Hs00950222_s1 | OR1L1 | 9 | Hs00537371_s1 | C9orf66 | 9 |
| Hs00979063_s1 | OR5C1 | 9 | Hs00273991_s1 | C9orf38 | 9 |
| Hs00979082_s1 | OR1J2 | 9 | Hs02379724_s1 | C9orf167 | 9 |
| Hs00946166_s1 | RPP38 | 10 | Hs00328927_s1 | C10orf71 | 10 |
| Hs01074992_s1 | FLJ40536 | 10 | Hs00740771_s1 | C10orf26 | 10 |
| Hs01102141_s1 | OR13A1 | 10 | Hs00744574_s1 | C10orf111 | 10 |
| Hs01119480_s1 | LOC100128295 | 10 | Hs00800009_s1 | C10orf58 | 10 |
| Hs00937357_s1 | KCNA4 | 11 | Hs00535489_s1 | C11orf71 | 11 |
| Hs00939787_s1 | OR10A3 | 11 | Hs00743006_s1 | C11orf34 | 11 |
| Hs00942596_s1 | OR8G5 | 11 | Hs00829922_s1 | C11orf51 | 11 |
| Hs00943966_s1 | OR5AP2 | 11 | Hs01876789_s1 | C11orf46 | 11 |
| Hs01081979_s1 | CMKLR1 | 12 | Hs00541466_s1 | C12orf12 | 12 |
| Hs01653110_s1 | KRT18 | 12 | Hs00703760_s1 | C12orf27 | 12 |
| Hs01656228_s1 | HNRNPA1 | 12 | Hs01000430_s1 | OR10AD1 | 12 |
| Hs01675517_s1 | OR10P1 | 12 | Hs01853597_s1 | C12orf47 | 12 |
| Hs00251199_s1 | CENPJ | 13 | Hs00937168_s1 | CYSLTR2 | 13 |
| Hs00703252_s1 | HS6ST3 | 13 | Hs01072517_s1 | SOX21 | 13 |
| Hs00705554_s1 | DLEU1 | 13 | Hs01635854_s1 | SIAH3 | 13 |
| Hs01057642_s1 | SOX1 | 13 | Hs01879077_s1 | UTP14C | 13 |
| Hs00745797_s1 | HSPA2 | 14 | Hs00544515_s1 | C14orf139 | 14 |
| Hs00746721_s1 | GLRX5 | 14 | Hs00740834_s1 | C14orf169 | 14 |
| Hs01040441_s1 | AKAP5 | 14 | Hs00762454_s1 | C14orf166 | 14 |
| Hs01083178_s1 | ADAM20 | 14 | Hs00952438_s1 | C14orf113 | 14 |
| Hs00534885_s1 | EID1 | 15 | Hs00257547_s1 | C15orf28 | 15 |
| Hs01045722_s1 | OR4F6 | 15 | Hs00258453_s1 | C15orf5 | 15 |
| Hs01098626_s1 | OR4F15 | 15 | Hs00611754_s1 | C15orf45 | 15 |
| Hs01921558_s1 | I5LR | 15 | Hs00752513_s1 | C15orf21 | 15 |
| Hs00990407_s1 | SSTR5 | 16 | Hs00536809_s1 | MGC16385 | 16 |
| Hs01874446_s1 | CHST4 | 16 | Hs00743683_s1 | ORAI3 | 16 |
| Hs01934174_s1 | PDP2 | 16 | Hs00752754_s1 | C16orf54 | 16 |
| Hs02515558_s1 | CHST6 | 16 | Hs00990408_s1 | SSTR5 | 16 |
| Hs00928342_s1 | FAM18B2 | 17 | Hs00362804_s1 | C17orf88 | 17 |
| Hs00962379_s1 | OR4D2 | 17 | Hs00536384_s1 | C17orf91 | 17 |
| Hs00990499_s1 | OR3A1 | 17 | Hs00753167_s1 | C17orf55 | 17 |
| Hs01004392_s1 | OR4D1 | 17 | Hs00760708_s1 | C17orf79 | 17 |
| Hs00252683_s1 | CHMP1B | 18 | Hs00260650_s1 | C18orf12 | 18 |
| Hs00255543_s1 | TCEB3B | 18 | Hs00537168_s1 | C18orf15 | 18 |
| Hs00535127_s1 | MEX3C | 18 | Hs00743508_s1 | C18orf32 | 18 |
| Hs00799523_s1 | ZNF271 | 18 | Hs00751979_s1 | C18orf25 | 18 |
| Hs01044607_s1 | VN1R1 | 19 | Hs00253883_s1 | C19orf28 | 19 |
| Hs01089409_s1 | OR7D2 | 19 | Hs00928195_s1 | S1PR5 | 19 |
| Hs01102536_s1 | GPR32 | 19 | Hs00980194_s1 | LOC100128439 | 19 |
| Hs01125256_s1 | OR1M1 | 19 | Hs01587883_s1 | C19orf42 | 19 |
| Hs00744406_s1 | RPS21 | 20 | Hs00274662_s1 | C20orf117 | 20 |
| Hs00921443_s1 | MOCS3 | 20 | Hs00329245_s1 | C20orf117 | 20 |
| Hs01920617_s1 | PRNP | 20 | Hs00708855_s1 | LOC100128496 | 20 |
| Hs01933675_s1 | PCMTD2 | 20 | Hs00708917_s1 | C20orf79 | 20 |
| Hs00270822_s1 | KCNE2 | 21 | Hs00257856_s1 | C21orf96 | 21 |
| Hs00273282_s1 | CLDN8 | 21 | Hs00897512_s1 | C21orf104 | 21 |
| Hs00937184_s1 | LRRC3 | 21 | Hs00964422_s1 | C21orf62 | 21 |
| Hs00942766_s1 | NRIP1 | 21 | Hs01022032_s1 | C21orf2 | 21 |
| Hs01934782_s1 | RTN4R | 22 | Hs00535829_s1 | C22orf29 | 22 |
| Hs01939563_s1 | SLC35E4 | 22 | Hs00703724_s1 | C22orf37 | 22 |
| Hs02379589_s1 | MGAT3 | 22 | Hs00704377_s1 | C22orf15 | 22 |
| Hs02512069_s1 | GSTT1 | 22 | Hs00739973_s1 | C22orf13 | 22 |
| Hs00918411_s1 | NGFRAP1 | X | Hs00542771_s1 | CXorf52 | X |
| Hs00937238_s1 | OTUD6A | X | Hs00702966_s1 | CXorf50 | X |
| Hs00961353_s1 | RPA4 | X | Hs00996628_s1 | CXorf52 | X |
| Hs01651394_s1 | USP51 | X | Hs01589213_s1 | CXorf27 | X |
| Hs00243216_s1 | SRY | Y | Hs00295373_s1 | CYorf15B | Y |
| Hs00371558_s1 | CDY1 | Y | Hs00245052_s1 | XKRY | Y |
| Hs00976796_s1 | SRY | Y | Hs00536782_s1 | TGIF2LX | Y |
| Hs01034378_s1 | NLGN4Y | Y | Hs00758603_s1 | XKRY | Y |

### Example 2

### Copy Number Assignment for 24 Chromosomes in a Trisomy 21 Female (47, XX +21)

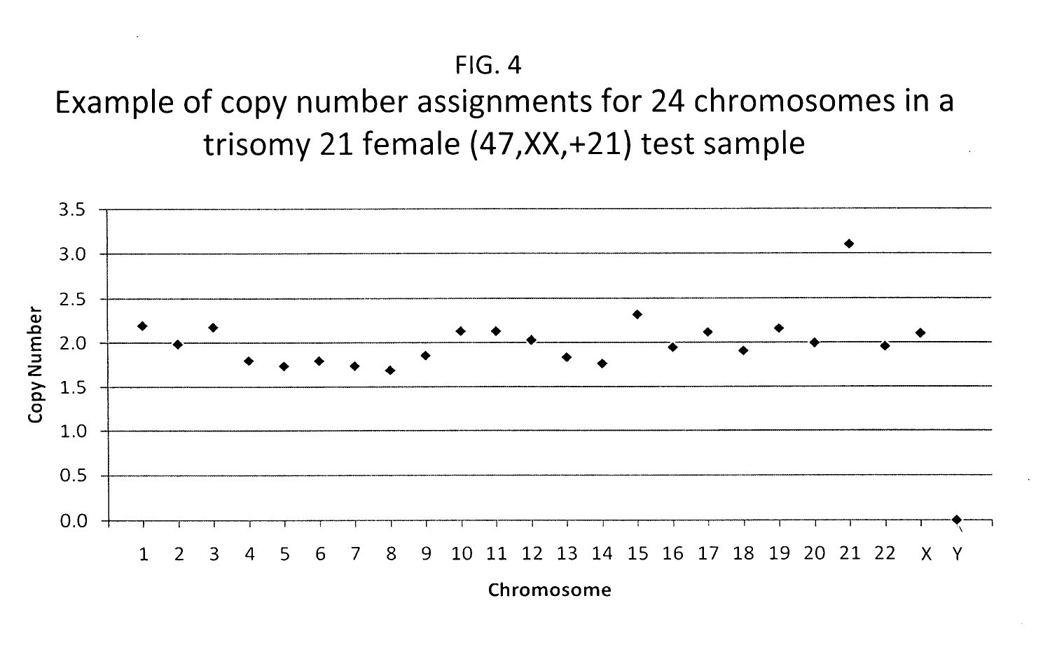

Employing the methods of the present invention using the 96 loci indicated in Table 7 (FAM assays), accurate copy number was able to be detected for all 24 chromosomes in an aneuploid cell line obtained from a trisomy 21 female (47, XX +21) (Coriell ID AG16777). In this experiment, 5 cells were lysed in alkaline lysis buffer, preamplified with 96 target loci assays (Table 7 FAM assays), and preamplified DNA was then loaded in quadruplicate into a Fluidigm BioMark RealTime PCR 96.96 Gene Expression Array according to the manufacturer's instructions (Fludigm Inc.) and using Gene Expression Master Mix (ABI). Normal female samples were run in parallel and the chromosome copy numbers were calculated as described here. Data are depicted herein in FIG. 4.

### Example 3

### PGD of an IVF Embryo may be Performed Using Invariant Loci and Real-time PCR and 2^{-ΔΔC}_{T} Analyses

It is contemplated that the invariant loci described in the Examples and Table 7 provided herein, as well as additional invariant loci that may be identified as disclosed, may be used to detect chromosome copy number in an IVF embryo using real-time PCR and 2^{-ΔΔC}_{T} analyses as outlined below:

As contemplated herein, embryos for PGD could undergo trophectoderm (TE) biopsy in the morning of day 5 or day 6 post-fertilization. A TE biopsy may be performed by conventional methods involving placing individual blastocysts into HTF-Hepes media (InVitro Care, Inc., Frederick, Md.) and opening a 5-10 μm hole in the zona pellucida, e.g., with a series of 1-3 single pulses from an infrared 1.48 μm diode laser utilizing a 1 millisecond single pulse duration at 100% power (Hamilton-Thorne Research, Beverly, Mass.). Herniating TE cells would then be aspirated into a trophectoderm biopsy pipette (Humagen, Charlottesville, Va.) and detached from the blastocyst by firing several pulses at the constricted area of TE cells at the end of the pipet. The biopsied piece of trophectoderm tissue would then be placed intact into a microcentrifuge tube after several washes through hypotonic solution. For example, for PGD of 10 embryos, this process would result in 10 PCR tubes, 1 for each embryo, and an extra tube in which an aliquot of the wash buffer would be loaded to serve as a negative control for possible contamination.

Each sample would then undergo lysis using standard procedures, e.g., using alkaline lysis methods familiar to one of skill in the art. For example, using this method, for a given sample, the sample volume would be brought to 8 μl with nuclease free water. One μl of potassium hydroxide lysis solution would then be added, and the sample would be mixed and incubated at 65° C. for 10 minutes. One μl of potassium chloride/Tris-HCl neutralization buffer would then be added and the sample mixed. (See, e.g., Cui et al., Proc. Natl. Acad. Sci USA Vol 86, pp 9389-9393, 1989 for detailed lysis and neutralization buffer recipes).

Referring to a set of invariant loci such as provided herein (e.g., as listed in Table 7 or otherwise previously determined as indicated herein), the lysate would then undergo preamplification of these targeted invariant loci using a pool of primer sets for each of those loci. Primer sets may be easily designed by one of skill in the art and/or obtained from commercial vendors, e.g., from Applied Biosystems Inc. The preamplification step would involve mixing the lysate with the primer pool, a DNA polymerase, and an appropriate reaction buffer (for example, PreAmp Cells-to-CT Master Mix (ABI)), and incubating the sample in a PCR thermalcycler, e.g., for 14, 16, or 18 cycles (or according to the recommendations of the reagent supplier).

Upon completion of the preamplification step, real-time PCR would then be performed on the samples. For example, each sample reaction could be aliquoted to real-time PCR reaction plate wells where real-time PCR reactions (e.g., 96 duplex wells) are set up in quadruplicate. Each duplex reaction could consist of two primer/probe sets, with one primer set and labeled probe (e.g., VIC labeled probe) that will produce fluorescence upon amplification that, is distinguishable from fluorescence derived from the second primer set and labeled probe (e.g., FAM labeled probe). Results for 96 duplex real-time PCR reactions in quadruplicate would be 768 real-time PCR curves (4 replicates×96 reactions×2 primer sets, 1 VIC and 1 FAM, per reaction) with signal intensities at each of 40 cycles. Data analysis software (such as ABI SDS or BioMark Real-Time PCR) could then be used to generate C_{T} values. The C_{T} value represents the cycle number for which a specific target sequence is amplified enough to reach an arbitrary threshold. (See, for example, FIG. 1). Premade primer sets that include premixed ready-to-use probes can be purchased from commercial providers such as Applied Biosystems TAQMAN Gene Expression Assays or Roche Applied Science Universal ProbeLibrary Reference Gene Assays. Alternatively, custom primers and probes can be developed manually through the use of primer design software familiar to one of skill in the art.

The resulting C_{T} values can then be exported to a data analysis program, e.g., Microsoft Excel or similar program which can be used to evaluate the data for each sample chromosome according to the calculations described above. (See, e.g. Table 1).

By way of example, calculations for the analysis of all 24 chromosomes, beginning with the analysis of chromosome 1, could be performed in an IVF embryo as follows:

First, the average of the C_{T}s determined for target chromosome 1 is calculated (“target chromosome average C_{T}”). (The actual number of total C_{T} values will depend on the number of loci assayed per chromosome.) Then the average of the C_{T}s for the remaining autosomes is determined (“endogenous control average C_{T}”). This is computed by adding all C_{T} values for all remaining autosomes and dividing by the number of remaining autosomal loci assays. Then the endogenous control average C_{T} is subtracted from the target chromosome average C_{T}. This value may be termed “the test sample ΔC_{T} for target chromosome 1”. The next step involves creating a ΔC_{T} from euploid reference samples previously evaluated in a manner similar to the procedure described above for the test sample ΔC_{T} for chromosome 1. Instead of only averaging C_{T}s from one reference sample, however, many reference samples can be used to improve accuracy. Thus, the reference sample ΔC_{T} for chromosome 1 could be the average of all C_{T}s for chromosome 1 assays for as many reference samples as possible or available at the time of embryo test sample analysis. Once the reference sample ΔC_{T} for chromosome 1 has been obtained it would then be subtracted from the test sample ΔC_{T} for chromosome 1. This value will be termed the “ΔΔC_{T} for the test sample for chromosome 1”. The ΔΔC_{T} for the test sample for chromosome 1 is then used as the negative exponent of 2 in order to compute fold change of chromosome 1 in the test sample relative to the reference sample(s). The chromosome 1 fold change is then multiplied by 2 in order to obtain the copy number of chromosome 1 in the test sample. In the case of analysis of all 24 chromosomes, this process is then repeated for chromosomes 2-22.

With regard to the sex chromosomes, the determination of the copy number for chromosome X or Y is performed slightly differently than the determination of copy number of autosomes. While the ΔC_{T} for chrX and Y is calculated in a manner identical to that described above for chr1-22, calculation of the ΔΔC_{T} for chrX and Y requires paying special attention to the type of reference sample used. For chrX, if the normal reference sample set is female, then the same protocol used for chr1 described above is adequate. However, if the normal reference set is male (1 copy of X) then the fold change of chrX is equal to the copy number of chrX and it does not need to be multiplied by 2. For chrY, the normal reference set has to be male and the fold change is equal to the copy number and doesn't need to be multiplied by 2.

The result of this series of analyses is a copy number for each of the 24 chromosomes of the test sample. This process would then be repeated as necessary so that each of the embryos to be analyzed for PGD would have copy number assignment for each chromosome. Copy number assignments would be evaluated in order to establish a diagnosis, e.g., of euploidy or monosomy, disomy, trisomy, for each chromosome in the IVF embryo and thus the embryo can be evaluated for the presence of a genetic defect.

As contemplated herein this entire process could be accomplished within 5 hours or less, thus, this procedure allows PGD to be performed on numerous embryos, followed by the same-day transfer of one or more embryos determined to be without genetic defect.

1. A method for preimplantation genetic diagnosis and fresh transfer of a day 3, day 4, day 5 or day 6 IVF embryo comprising
(a). performing real-time PCR and 2^{-ΔΔC}_{T} analyses to determine normalized copy number of at least one invariant locus in the embryo on at least one chromosome of the IVF embryo;
(b). determining the presence or absence of a genetic defect in the embryo based on the normalized copy number of the invariant loci in the embryo; and
(c). transferring the embryo if determined to be without genetic defect within about 24 hours of performing step (a).

2. The method of claim 1 wherein said genetic defect is aneuploidy.

3. The method of claim 2 wherein the aneuploidy is selected from the group consisting of nullisomy, monosomy, disomy, trisomy, and tetrasomy.

4. The method of claim 1 wherein said IVF embryo is also screened for a genetic defect that is not aneuploidy.

5. The method of claim 4 wherein the genetic defect is selected from the group consisting of those provided in Table 2.

6. A method for transferring an IVF embryo comprising
(a). performing real-time PCR and 2^{-ΔΔC}_{T} analyses to determine the presence or absence of a genetic defect in the embryo based on normalized copy number of at least one invariant locus on at least one chromosome collected from at least one cell of the embryo; and
(b). transferring the embryo if determined to be without genetic defect within about 154 hours of fertilization.

7. The method of claim 6 wherein the embryo is transferred between about 48 and about 144 hours of fertilization.

8. The method of claim 6 wherein the performing and transferring steps are accomplished within a period of about 48 hours.

9. The method of claim 6 wherein the performing and transferring steps are accomplished within a period of about 24 hours.

10. The method of claim 6 wherein the performing and transferring steps are accomplished within a period of about 16 hours.

11. The method of claim 6 wherein the performing and transferring steps are accomplished within a period of about 12 hours.

12. The method of claim 6 wherein the performing and transferring steps are accomplished within a period of about 8 hours.

13. The method of claim 6 wherein the performing and transferring steps are accomplished within a period of about 5 hours.

14. The method of claim 6 wherein said genetic defect is aneuploidy.

15. The method of claim 14 wherein the aneuploidy is selected from the group consisting of nullisomy, monosomy, disomy, trisomy, and tetrasomy.

16. The method of claim 6 wherein said IVF embryo is also screened for a genetic defect that is not aneuploidy.

17. The method of claim 16 wherein the genetic defect is selected from the group consisting of those provided in Table 2.

18. A method for determining the presence or absence of a genetic defect in an IVF embryo comprising:
(a). performing real-time PCR and 2^{-ΔΔC}_{T} analyses to determine normalized copy number of at least one invariant locus on at least one chromosome collected from at least one cell of the embryo and
(b). selecting a candidate IVF embryo determined to be without genetic defect.

19. The method of claim 18 wherein said genetic defect is aneuploidy.

20. The method of claim 19 wherein the aneuploidy is selected from the group consisting of nullisomy, monosomy, disomy, trisomy, and tetrasomy.

21. The method of claim 18 wherein said IVF embryo is also screened for a genetic defect that is not aneuploidy.

22. The method of claim 21 wherein the genetic defect is selected from the group consisting of those provided in Table 2.

23. The method of claim 18 wherein determining the presence or absence of a genetic defect in the embryo comprises copy number analysis of at least one invariant locus on all of the chromosomes of the embryo.

24. The method of claim 18 wherein the IVF embryo is a human embryo.

25. The method of claim 18 wherein the IVF embryo is a day 3, day 4, day 5 or day 6 embryo.

26. The method of claim 18 wherein step (b) is performed within 3-6 days of in vitro fertilization of said embryo.

27. The method of claim 18 wherein the invariant loci are located on chromosomes selected from the group consisting of chromosomes 1, 2, 3, 4, 5, 6, 7, 8, 9, 10, 11, 12, 13, 14, 15, 16, 17, 18, 19, 20, 21 22, X, and Y.

28. The method of claim 27 wherein the chromosomes are chromosomes 13, 18, and 21.

29. The method of claim 18 further comprising transferring the selected candidate IVF embryo on the same day as the steps of performing and selecting.

30. The method of claim 29 wherein performing, selecting and transferring of the IVF embryo are accomplished within about 12 hours or less.

31. The method of claim 29 wherein performing, selecting and transferring of the IVF embryo are accomplished within about 8 hours or less.

32. The method of claim 29 wherein performing, selecting and transferring of the IVF embryo are accomplished within about 5 hours or less.

33. The method of claim 29 wherein the IVF embryo is a blastocyst.

34. The method of claim 33 wherein the cells are biopsied from trophectoderm.

35. The method of claim 33 wherein performing, selecting and transferring of the blastocyst are accomplished within about 24 hours or less.

36. The method of claim 33 wherein performing, selecting and transferring of the blastocyst are accomplished within about 12 hours or less.

37. The method of claim 33 wherein performing, selecting and transferring of the blastocyst are accomplished within about 8 hours or less.

38. The method of claim 33 wherein performing, selecting and transferring of the blastocyst are accomplished within about 5 hours or less.

39. The method of claim 29 wherein three or less IVF embryos are transferred.

40. The method of claim 29 wherein two or less IVF embryos are transferred.

41. The method of claim 29 wherein one IVF embryo is transferred.

42. The method of claim 18, wherein determining the presence or absence of a genetic defect in the embryo is based on the copy number of about 100 or less invariant loci per chromosome.

43. The method of claim 18, wherein determining the presence or absence of a genetic defect in the embryo is based on the copy number of about 50 or less invariant loci per chromosome.

44. The method of claim 18, wherein determining the presence or absence of a genetic defect in the embryo is based on the copy number of about 40 or less invariant loci per chromosome.

45. The method of claim 18, wherein determining the presence or absence of a genetic defect in the embryo is based on the copy number of about 20 or less invariant loci per chromosome.

46. The method of claim 18, wherein determining the presence or absence of a genetic defect in the embryo is based on the copy number of at least two invariant loci.

47. The method of claim 18, wherein determining the presence or absence of a genetic defect in the embryo is based on the copy number of at least three invariant loci.

48. The method of claim 18, wherein determining the presence or absence of a genetic defect in the embryo is based on the copy number of at least five invariant loci.

49. The method of claim 18, wherein determining the presence or absence of a genetic defect in the embryo is based on the copy number of at least ten invariant loci.

50. An array comprising a plurality of nucleic acid probes comprising nucleic acid for at least one invariant locus from at least one human chromosome.

51. The array of claim 50 wherein the probes are immobilized on a solid support.

52. The array of claim 50 wherein the nucleic acid in the array comprises at least two invariant loci from at least one of human chromosomes 1-22, X and Y.

53. A method for making an array for preimplantation genetic diagnosis of an IVF embryo comprising (a) identifying at least one invariant locus for preimplantation genetic diagnosis, (b) selecting at least one invariant locus for at least one chromosome, and (c) affixing nucleic acid probes for the invariant loci on a solid support.

54. The method of claim 53 wherein from about one to about 100 invariant loci for at least one chromosome are selected.

55. A kit comprising an array of nucleic acid probes immobilized on a solid support, the array comprising nucleic acid probes for at least one invariant locus from at least one human chromosome wherein the invariant loci are useful for determining the presence or absence of a genetic defect in an IVF embryo prior to transfer according to the method of claim 18.
